(19) United States
(12) Patent Application Publication
(10) Pub. No.: US 2023/0200834 A1
(43) Pub. Date: Jun. 29, 2023

(54) ULTRASONIC TOOL AND METHOD FOR MANUFACTURING THE TOOL

(71) Applicant: BOSONIC AG, Bern (CH)

(72) Inventors: Loïc Sottas, Lausanne (CH); Laurent Torriani, Lamboing (CH); Marcel Aeschlimann, Ligerz (CH); Antoine Bonadei, Bienne (CH)

(21) Appl. No.: 17/925,620

(22) PCT Filed: May 18, 2021
(86) PCT No.: PCT/EP2021/063044

§ 371 Date: Nov. 16, 2022

(30) Foreign Application Priority Data
May 18, 2020 (CH) 00595/20

(51) Int. Cl. A61B 17/32; A61B 34/30; A61B 34/00

(52) U.S. Cl. CPC A61B 17/320068; A61B 34/30; A61B 34/77; A61B 2017/00084; A61B 2017/320077; A61B 2017/320084

(57) ABSTRACT

An ultrasonic cutting tool for use in an ultrasonic instrument, the tool being a blade manufactured from a tubular blank by flattening a section of the blank. The blade includes a flat section designed to be used for cutting, a tubular section, and a transition section joining the flat section to the tubular section. The tubular section is joined to or includes an attachment section for attaching the blade to an ultrasonic generator and serving as a conduit to bring a cooling fluid to the distal end or to suck particles into the hand tool.

### BACKGROUND OF THE INVENTION

### Field of the Invention

The invention relates to the field of ultrasonic instruments. It relates to an ultrasonic tool for use in an ultrasonic instrument, in particular for cutting or abrasive machining, and to a method for manufacturing the tool.

### Description of Related Art

Ultrasonic instruments have a generator of ultrasonic energy and an elongated tip or tool or blade, whose proximal end receives the ultrasonic energy from the generator and conveys it to the tip distal end. Depending on the application, the distal end can be shaped for the instrument to be used as a probe and/or for penetrating soft tissue, cutting or working bone tissue etc.

It is known to provide ultrasonic tools with conduits for cooling fluid. The fluid can be water or a mixture of water with ethanol and/or a disinfectant fluid. The fluid is used to cool down the blade and to rinse away the cut material. Conduits can be double walled in order to provide for an additional conduit for aspiring fluid material, as shown in US 4515583 and US 6165150 A. Furthermore, it is known to have cooling water channels that branch out to a plurality if exit openings, as in US 5188102 or to be made of a porous, sintered material to allow water to exit through the surface of an ultrasonic cutting blade, as in US 2015/0005774 A1. To have an efficient cooling of the blade during bone cutting is still a challenge today. In those solutions, the cooling fluid send out a mist around the blade which disturbs the field of view of the surgeon while operating.

US5836595 discloses an ultrasonic tip for intraocular surgery, also called phaco tip, for Phacoemulsification. It has a flat end created by crimping a cylindrical tube.

US2019254731 shows an ablation pad for heart ablation. Therein, two plates can be welded together, with the welding lines forming walls and channels for a fluid medium.

WO2018220515 shows an ultrasonic cutting device for osteotomy with a threaded connection between the cutter and an extension rod which in turn is attached to a handpiece.

These devices are generally manufactured by elaborate or time-consuming processes such as machining or sintering, which make them costly. There is a need for ultrasonic cutting implements of simple construction that can be manufactured efficiently and economically.

### SUMMARY OF THE INVENTION

It is therefore an object of the invention to create an ultrasonic tool of the type mentioned initially, which overcomes the disadvantages mentioned above. A further object is to create a method for manufacturing the tool.

One advantage is that coolant and cleaning fluid is supplied from the inside of the tool. This avoids producing large jets of water in the working area, and thereby can improve visibility in the working area.

The ultrasonic cutting tool, for use in an ultrasonic instrument, is a blade manufactured from a tubular blank by flattening a section of the blank, the blade including
a flat section designed to be used for cutting,
a tubular section and
a transition section joining the flat section to the tubular section,
the tubular section being joined to or including an attachment section for attaching the blade to an ultrasonic generator.

The tool can be used in an ultrasonic instrument, coupled to an ultrasonic vibration generator, as a blade for cutting or abrasive machining. The tool can be cooled from the inside, by passing a coolant through the tool along the longitudinal direction. Conversely, material can be sucked from the vicinity of the tool. The flat section can be manufactured to form a cutting tool. In particular, its flat surface can be shaped to constitute a file or a rasp, and/or edges of the flat section can be shaped to constitute a file or rasp or a knife.

In embodiments, the flat section forms one or more channels suited to guide a fluid along the inside of the blade. The channels can transport cooling fluid to the distal end of the blade and/or can suck liquid or particles through the blade.

This allows to guide cooling fluid through and along the inside of the blade and in particular the flat section. This in turn can serve to cool the blade and its surroundings. Compared to external cooling, visibility in the work area is better.

The fluid being guided through the attachment region and inside the flat section makes the tool particularly suited for use in combination with a robot holding and moving the tool, since extraneous tubes or hoses for providing coolant are eliminated.

Shaping the flat section and tubular section from the same blank allows to create tools with a variety of lengths, and in particular relatively long tools in a simple manner. The length of the tool can be, for example, between 20 mm and 200 mm, in particular between 40 mm and 150 mm, in particular between 80 mm and 120 mm.

In embodiments, the flat section includes one or more holes in fluid communication with the one or more channels.

In embodiments, one or more edges of the flat section include teeth.

In embodiments, one or more edges of the flat section are machined to constitute a cutting edge.

In embodiments, one or more edges of the flat section include one or more notches constituting openings that are in liquid communication with the one or more channels.

In embodiments, an outer surface of the flat section is shaped to include a structured surface, in particular with teeth or grooves. The structured surface can act as a file.

The presence of the teeth and/or cutting edge and/or notches and/or structured surface and/or holes can improve the efficiency of cutting and/or abrading. In particular, edges of the holes can participate in cutting and/or abrading. The holes and/or notches being in liquid communication with a channel serves to guide liquid to where the cutting and/or abrading takes place, and where the cooling effect can be most needed.

In embodiments, the flat section includes one or more weld lines, in particular running along a longitudinal direction in which the flat section extends.

The one or more weld lines can create separate longitudinal channels between them.

In embodiments, the attachment section includes an internal thread or an external thread.

In embodiments, the attachment section includes a longitudinal conduit in liquid communication with the inside of the tubular section.

The longitudinal conduit can serve for guiding fluid through the attachment section into or out of an inside of the tubular section.

The method for manufacturing the tool according to one of the preceding claims, wherein the method includes:
providing a tubular blank;
pressing a distal end of the blank to form the flat section, leaving a proximal end of the blank in a tubular shape, constituting the tubular section;
machining the proximal end of the blank to form the attachment section; or attaching an attachment element to the proximal end to form the attachment section.

The attachment section can be formed before or after pressing the distal end.

In embodiments, the structured surface of the flat section is created, and/or one or more holes are created and/or one or more notches or teeth are created before the flat section is formed.

In embodiments, the structured surface of the flat section is created, and/or one or more holes are created and/or one or more notches or teeth are created after the flat section is formed.

In embodiments, the structured surface and/or one or more holes and/or one or more notches or teeth are created in the process of flattening the blank to form the flat section.

In embodiments, the flattened tube is bent to form a curved flattened tube.

In embodiments, the contour of the flattened tube, in particular its distal end, is shaped to a non-rectangular contour. For example, it can be rounded, tapering, have a split contour with two distal end points, etc. This can provide the tool with further functionality.

According to an aspect of the invention, a robotic system is provided, configured to be equipped with a cutting tool as described herein, the robotic system being programmed to apply the tool to machine an object or workpiece.

In embodiments, the workpiece is a piece of animal or human tissue, in particular bone.

In embodiments, the robotic system is configured to supply the cutting tool with a fluid coolant while machining the workpiece.

The tool being internally cooled allows for a continuous cooling of the tool in a more efficient manner and with a better control of the cooling and thus of the temperature of the tool. This in turn allows for longer machining time windows.

Longer machining time windows in turn allow for machining the workpiece without withdrawing the tool, which would otherwise re-inserting the tool, leading a loss of precision. Furthermore, different functions using the same tool can be implemented without withdrawing the tool. Such functions can be cutting, sawing filing, cooling, and aspiration of material.

Combination of the tool with a robot manipulator allows for a controlled cutting or machining of three-dimensional cuts and shapes, respectively.

In embodiments, the robotic system includes a manipulator arm to which the tool is attached and by which the tool is movable, and wherein the tool is provided with cooling fluid through the manipulator arm, in particular wherein a cooling fluid conduit is arranged inside a casing of at least one most distal link of the manipulator arm.

In embodiments, the robotic system is programmed to apply the tool to machine the workpiece in an uninterrupted sequence, without withdrawing the tool from a region in which it is applied to the workpiece.

In embodiments, the robotic system is programmed to apply the tool to machine the workpiece using two or more different functions of the tool without withdrawing the tool, in particular wherein the functions are cutting, sawing filing, and aspiration of material.

In embodiments, the robotic system is programmed to apply the tool to machine different sides of the workpiece, in particular surfaces of the workpiece whose surface normal are oriented at an angle of more than forty-five degrees or more than ninety degrees relative to one another.

That is, the tool is used to machine two or more different sides of the workpiece.

In embodiments, the robotic system is programmed to apply the tool to machine the workpiece in an uninterrupted sequence of at least two minutes or three minutes or four minutes or five or six minutes.

In embodiments, the tool is shaped to include the function of at least two of a file, a saw, or a knife.

For example, the tool can include a file and a saw, or a saw and a knife, etc. This allows to apply the tool without the need to interrupt the machining operation and to withdraw the tool.

In embodiments, the tool is shaped to include at least two variants of the same function but with different parameters.

For example, the tool can include a rough file and a fine file, or a rough saw and a fine saw.

In embodiments, the robotic system includes a sensing unit configured to measure a tool force exerted by the tool on the workpiece, and being configured to control a movement of the tool according to the measured tool force.

This makes it possible to control the movement of the tool in order to maintain a desired machining force. This in turn can be used to optimise machining speed and/or prevent excessive heating of the tool.

In embodiments, the robotic system includes a coolant supply unit configured to provide cooling fluid to the tool intermittently, in particular with first time durations in which cooling fluid is provided alternating with second time durations in which no cooling fluid is provided, in particular wherein a time period after which the first time durations occur lies between one and ten seconds, in particular between two and five seconds.

In other words, the first time durations in which cooling fluid is provided correspond to pulses of cooling fluid, and the pulses can be repeated with a period length according to the time period.

The flow of cooling fluid being intermittent prevents a cushion of fluid being created and maintained between the tool and the workpiece and thereby impairing operation of the tool. During a cooling fluid pulse, debris from the tool’s operation can be washed away.

In embodiments, the robotic system or the coolant supply unit includes a sensing unit configured to measure a tool temperature and a control unit configured to control a flow of cooling fluid to the tool according to the measured tool temperature.

This allows to adapt the flow of coolant to the actual cooling requirements, which in turn depend on the working condition between the tool and the workpiece.

Controlling the flow can be done by continuously varying the flow, or with discrete steps, in particular by turning the flow on and off, that is, by a pulsating flow. In the latter case, the controller can set a pulse width, or a pulse frequency or coolant pulses.

In embodiments, the sensing unit is configured to determine the tool temperature on the basis of a driver frequency of oscillation of the tool, the driver frequency of oscillation being continuously adapted to an actual resonance frequency of the tool.

This is based on the observation that the temperature of the tool affects the mechanical properties of the tool, in particular its length, and thereby an actual resonance frequency of the tool. The actual resonance frequency can be determined by using an ultrasound driver that automatically adapts its operating frequency to the actual resonance frequency of the tool. This automatic frequency adaptation is a feature of many existing ultrasound drivers.

As a result, the flow of coolant can be controlled according to the actual operating frequency of the ultrasound driver.

Providing cooling fluid to the tool intermittently, and/or controlling the flow of fluid and or measuring the temperature as described herein can also be implemented by means of a coolant supply unit that is part of a setting in which no robotic system is present.

### BRIEF DESCRIPTION OF THE DRAWINGS

The subject matter of the invention will be explained in more detail in the following text with reference to exemplary embodiments which are illustrated in the attached drawings, which schematically show:

FIG. 1 a perspective view of a blade;

FIG. 2 a longitudinal cross section of the blade;

FIGS. 3-4 a transverse section and an elevated view of a flat section of the blade according to one embodiment.

FIGS. 5-8 sections and elevated views of further embodiments;

FIGS. 9-10 further embodiments in an elevated view; and

FIG. 11 yet a further embodiment in a cross section.

### DETAILED DESCRIPTION OF THE INVENTION

In principle, identical parts are provided with the same reference symbols in the figures.

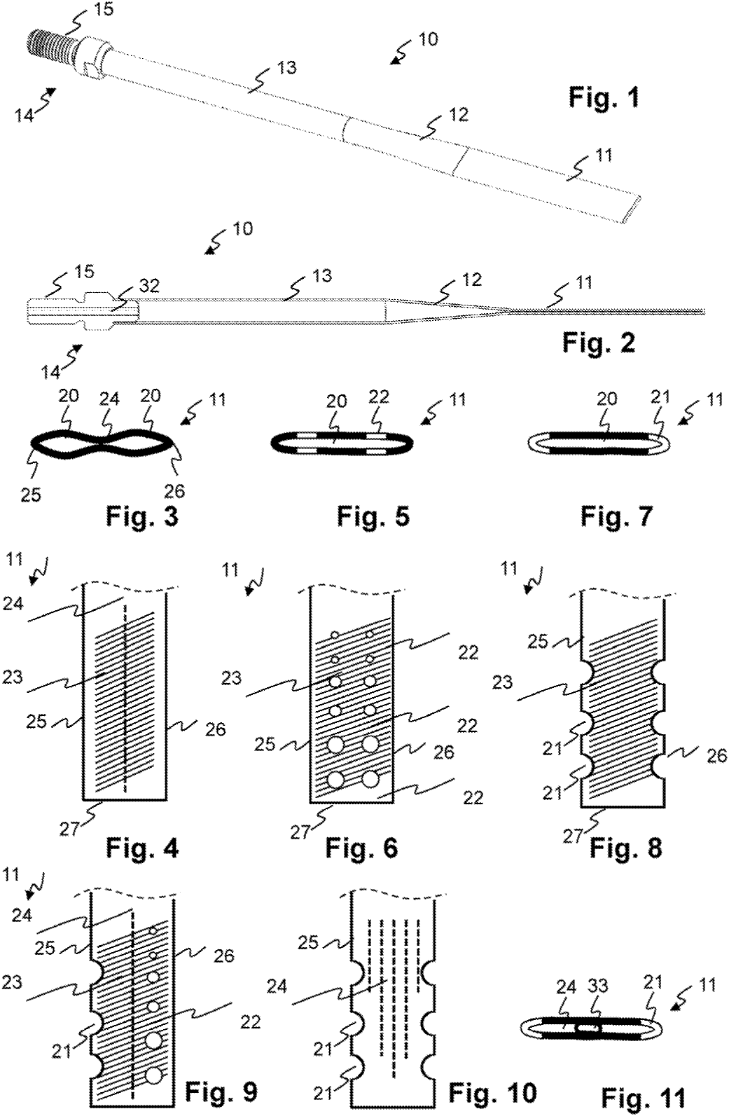

FIG. 1 shows a perspective view of a blade 10, and FIG. 2 a longitudinal cross section of the blade. The blade 10 includes a flat section 11, being the working section, connected via a transition section 12 to a tubular section 13, which in turn is connected to an attachment section 14. The flat section 11, transition section 12, tubular section 13 and optionally also the attachment section 14 can be formed from a single tubular blank.

In the flat section 11, the former lumen of the tube forms a longitudinal channel 20, which can be used to guide, distribute and dispense coolant provided via the tubular section 13. A longitudinal conduit 32 is arranged to supply coolant through the attachment section 14 to the tubular section 13.

By means of the attachment section 14, the blade 10 can be attached to an ultrasonic vibration generator, for example by means of an outer thread 15, as shown, or an inner thread.

The transporting and dispensing of the fluid can be enhanced or facilitated by a pumping effect caused by ultrasonic oscillations of the blade 10 and in particular the flat section 11 and/or the transition section 12.

FIGS. 3-4 show a transverse section and an elevated view of a flat section of the blade 10 wherein a longitudinal weld line 24 creates separate channels 20. The principal surface of the flat section 11 includes structured surfaces 23 such as teeth or grooves. The 8-shape of the transverse section can be obtained when flattening the tube, even if it is not welded afterwards.

Generally, a first longitudinal edge 25, second longitudinal edge 26 and front edge 27 can be shaped differently or in the same way, with notches 21, teeth, serrations or as blades, or with a combination of these and even other elements.

FIGS. 5-6 show a transverse section and an elevated view of a flat section of the blade 10 wherein holes 22 are present, constituting openings to the channel 20. Edges of the holes can have a cutting effect. The diameter of the holes 22 is varied in the longitudinal direction in order to control the distribution of the flow of coolant along the length of the flat section 11. This can serve to evenly distribute the flow.

FIGS. 7-8 show a transverse section and an elevated view of a flat section of the blade 10 wherein notches 21 are present in one or more of the first edge 25 and/or second edge 26 and/or front edge 27. The notches 21 serve on the one hand as serrations for cutting, and on the other hand as conduits guiding coolant out of the channel 20.

FIGS. 3 to 8 show elements such as weld line 24, structured surface 23, holes 22 and notches 21 separately. In other embodiments, they are combined. For example, according to FIG. 9, notches 21 are present at a first edge 25, holes 22 are arranged near a second edge 26, and the second edge 26 can be shaped as a cutting edge.

In further embodiments, two or more weld lines 24 are present, creating a corresponding number of channels 20 between them. Weld lines 24 can be used to stiffen the structure of the flat section 11 and thereby modify its natural frequency of oscillation. FIG. 10 shows weld lines 24 distributing coolant to outlets, which in this case are notches 21.

FIG. 11 shows a further embodiment, with an inner tube 33 that originally was arranged concentrically in the blank and after flattening constitutes a separate longitudinal channel in the blade 10 and in particular in the flat section 11.

Cutouts such as holes 22 and notches 21 can be machined, for example, by stamping or laser cutting. Other, smaller structures, such as the structured surface 23, can be created by laser engraving or etching. Cutouts and the other structures can be created on the blank, before flattening the blank to form the flat section 11, or afterwards.

The structured surface 23 and/or the notches 21 can be created in the process of flattening the blank to form the flat section 11.

The blade 10 can include separate channels. The separate channels can be used for evenly distributing coolant along the flat section 11. Alternatively or in addition, they can be used for different purposes: at least one coolant channel can be used for providing coolant to the flat section 11, and at least one suction channel can be used for sucking material from the region surrounding the flat section 11.

Separate channels can be formed by one or more weld lines 24 that join opposite sections of the flat section 11, as shown in FIG. 3. Separate channels can be formed by an inner tube 33 arranged inside the blade 10 and extending in its longitudinal direction, as shown in FIG. 11.

In a specific embodiment, a stainless steel tube with an outer diameter of 4 millimetres and an inner diameter of about 3.5 millimetres is connected with a press fit to an attachment section 14 made of titanium. The attachment section 14 has a thread 15 of dimension M4, that is, with an outer diameter of 6 millimetres. The overall length of the blade 10 is about 100 millimetres, the thickness of the flat section 11 is about 0.9 millimetres. The blade 10 can be operated with an ultrasound driver having an operating frequency of 26 kHz. The blade itself has a resonance frequency of about 26 kHz.

This frequency relates to longitudinal oscillations, thus oscillations in the direction of the longitudinal axis of the blade 10 as a whole and the flat section 11 in particular.

While the invention has been described in present embodiments, it is distinctly understood that the invention is not limited thereto, but may be otherwise variously embodied and practised within the scope of the claims.

1. An ultrasonic cutting tool, for use in an ultrasonic instrument, the tool being a blade manufactured from a tubular blank by flattening a section of the blank, the blade comprising:
a flat section configured to be used for cutting,
a tubular section, and
a transition section joining the flat section to the tubular section,
the tubular section being joined to or comprising an attachment section for attaching the blade to an ultrasonic generator.

2. The tool of claim 1, wherein the flat section forms one or more channels suited to guide a fluid along the inside of the blade.

3. The tool of claim 1, wherein the flat section comprises one or more holes in fluid communication with the one or more channels.

4. The tool of claim 1, wherein one or more edges of the flat section comprise teeth.

5. The tool of claim 1, wherein one or more edges of the flat section are machined to constitute a cutting edge.

6. The tool of claim 1, wherein one or more edges of the flat section comprise one or more notches constituting openings that are in liquid communication with the one or more channels.

7. The tool of claim 1, wherein an outer surface of the flat section is machined to comprise a structured surface with teeth or grooves.

8. The tool of claim 1, wherein the flat section comprises one or more weld lines, in particular running along a longitudinal direction in which the flat section extends.

9. The tool of claim 8, wherein the one or more weld lines create separate longitudinal channels in a direction in which the flat section extends.

10. The tool of claim 8, wherein the one or more weld lines are arranged to distribute coolant flowing in a direction of a distal end of the flat section towards a side of the flat section.

11. The tool of claim 1, wherein the attachment section comprises an internal thread or an external thread.

12. The tool of claim 1, wherein the attachment section comprises a longitudinal conduit in liquid communication with an inside of the tubular section.

13. Method for manufacturing the blade according to claim 1, wherein the method comprises the steps of:
providing a tubular blank;
pressing a distal end of the blank to form the flat section, leaving a proximal end of the blank in a tubular shape, constituting the tubular section;
machining the proximal end of the blank to form the attachment section, or attaching an attachment element to the proximal end to form the attachment section.

14. The method of claim 13, wherein a surface of the flat section is machined, and/or one or more holes are created and/or one or more notches are created before the flat section is formed.

15. The method of claim 13, wherein a surface of the flat section is machined, and/or one or more holes are created and/or one or more notches are created after the flat section is formed.

16. A robotic system, configured to be equipped with a cutting tool according to claim 1, the robotic system being programmed to apply the tool to machine an object or workpiece.

17. The robotic system according to claim 16, comprising a manipulator arm to which the tool is attached and by which the tool is movable, and wherein the tool is provided with cooling fluid through the manipulator arm, in particular wherein a cooling fluid conduit is arranged inside a casing of at least one most distal link of the manipulator arm.

18. The robotic system according to claim 16, programmed to apply the tool to machine the workpiece in an uninterrupted sequence, without withdrawing the tool from a region in which it is applied to the workpiece.

19. The robotic system according to claim 18, programmed to apply the tool to machine the workpiece using two or more different functions of the tool without withdrawing the tool, in particular wherein the functions are cutting, sawing filing, and aspiration of material.

20. The robotic system according to claim 16, programmed to apply the tool to machine different sides of the workpiece, in particular surfaces of the workpiece whose surface normal are oriented at an angle of more than forty-five degrees or more than ninety degrees relative to one another.

21. The robotic system according to claim 16, programmed to apply the tool to machine the workpiece in an uninterrupted sequence of at least two minutes or three minutes or four minutes or five or six minutes.

22. The robotic system according to claim 16, wherein the tool is shaped to comprise the function of at least two of a file, a saw, or a knife.

23. The robotic system according to claim 22, wherein the tool is shaped to comprise at least two variants of the same function but with different parameters.

24. The robotic system according to claim 16, comprising a sensing unit configured to measure a tool force exerted by the tool on the workpiece, and being configured to control a movement of the tool according to the measured tool force.

25. The robotic system according to claim 16, configured to provide cooling fluid to the tool intermittently, in particular with first time durations in which cooling fluid is provided alternating with second time durations in which no cooling fluid is provided, in particular wherein a time period after which the first time durations occur lies between one and ten seconds, in particular between two and five seconds.

26. The robotic system according to claim 16, comprising a sensing unit configured to measure a tool temperature and a control unit configured to control a flow of cooling fluid to the tool according to the measured tool temperature.

27. The robotic system according to claim 26, wherein the sensing unit is configured to determine the tool temperature on the basis of a driver frequency of oscillation of the tool, the driver frequency of oscillation being continuously adapted to an actual resonance frequency of the tool.

28. Robotic system according to claim 16, configured to control a flow of cooling fluid to the tool according to an actual operating frequency of the ultrasound driver.
